(19) United States
(12) Patent Application Publication
(10) Pub. No.: US 2012/0214892 A1
(43) Pub. Date: Aug. 23, 2012

(54) PROCESS FOR PRODUCING POLYESTERETHEROLS

(75) Inventors: Andreas Kunst, Ludwigshafen (DE); Sirus Zarbakhsh, Hong Kong (CN); Gunnar Kampf, Stemwede-Haldem (DE); Marco Balbo Block, Osnabrueck (DE)

(73) Assignee: BASF SE, Ludwigshafen (DE)

(21) Appl. No.: 13/397,163

(22) Filed: Feb. 15, 2012

Related U.S. Application Data
(60) Provisional application No. 61/443,753, filed on Feb. 17, 2011.

(51) Int. Cl. C08G 18/28; C07C 69/78

(52) U.S. Cl. 521/173; 560/73

(57) ABSTRACT

The present invention relates to a novel process for producing polyesteretherols via alkoxylation of polyesterols, and also to the use of the polyesteretherols for producing polyurethanes.

The present invention relates to a novel process for producing polyesteretherols via alkoxylation of polyesterols, and also to the use of the polyesteretherols for producing polyurethanes.

The production of polyetherester polyols is known. In DE 36 13 875, for example, a process is described for producing polyesterether polyols where production of the polyester polyols preferably uses aliphatic carboxylic acids, and alkoxylation of the intermediate preferably uses ethylene oxide.

The alkoxylation process here is carried out at relatively high temperatures of about 160° C., and this can lead to side reactions.

The use of esters for improving the flame retardancy of rigid PIR (polyisocyanurate) foams and rigid PU (polyurethane) foams has likewise been disclosed and described. Systems of this type differ from traditional PO-ether-based rigid PU foams in having increased reactivity and poor adhesion; this can be borderline in the case of ester-based rigid PU foams. An adhesion promoter is therefore often used to improve adhesion in such systems, as described in EP 1516720 A1. Furthermore, specifically in the case of PIR systems, the temperature of the twin-belt system during processing has to be high, in order to achieve adequate adhesion and curing.

The use of polyesters in relatively high-density, glass fiber reinforced rigid polyurethane foams is also known, and has been described for example in WO2010066635. Here, the liquid polyurethane reaction mixture is applied to a plurality of glass fiber mat layers. The reaction mixture first has to penetrate completely into the glass fiber layers, before it begins to foam. This penetration time is critical for homogeneous distribution of the glass fibers in the foam, and also for maximum achievable foam production rate.

An object of the present invention was therefore to develop polyesteretherols which are intended for flame-retardant, ester-based, preferably Br-free rigid PIR and PU foams, and which have reduced brittleness, and which can be processed at usual twin-belt temperatures without adhesion promoter while nevertheless achieving adequate adhesion. The rigid foams are moreover intended to exhibit better hardening and therefore higher dimensional stability on leaving the twin-belt system.

Another object was moreover to develop polyesteretherols which are intended for glass fiber reinforced rigid PU foams and which lead to a liquid polyurethane reaction mixture which penetrates markedly more rapidly into glass fiber layers and thus permits faster production of the reinforced rigid foams with identical foam quality, in particular based on the homogeneous distribution of the glass fibers in the foam.

The object was then achieved by using base catalysis to alkoxylate polyesterols made of certain aromatic dicarboxylic acids (or derivatives of these) with propylene oxide (PO) or with a mixture made with propylene oxide and ethylene oxide (EO).

Surprisingly, it has been demonstrated that the use of propoxylated esters does not lead to a reduction of the reactivity of the systems; instead, a marked improvement is actually achieved in the hardening process and in adhesion to the outer layers, and the systems can be processed at usual twin-belt temperatures of less than 60° C.

The present invention therefore provides a process for producing a polyesteretherol, where at least one compound selected from the group consisting of aromatic dicarboxylic acids, anhydrides of these, and mixtures thereof is first reacted with at least one compound from the group of the polyhydric alcohols to form a polyesterol A with an acid number smaller than 5 mg KOH/g, and then, with use of a basic catalyst, the resultant polyesterol A is reacted with propylene oxide or with a mixture made with propylene oxide and ethylene oxide.

The present invention further provides a polyesteretherol which can be produced by the process of the invention, and also the use, for producing a polyurethane, of a polyesteretherol which can be produced by the process of the invention.

The present invention further provides a rigid polyurethane foam which is obtainable via mixing of compounds having groups reactive toward isocyanates, and of further components (blowing agent, flame retardant, catalysts, and further additives), where the compounds having groups reactive toward isocyanates include at least 5% by weight of a polyesteretherol which can be produced by the process of the invention, based on the total weight of the compounds having groups reactive toward isocyanates.

For the purposes of the present invention, the term “polyesterol” is equivalent to the expression “polyester alcohol”, and designates a polyester having an average of at least 1.5 free alcohol groups and having an acid number lower than 5 mg KOH/g of polyesterol, preferably lower than 2 mg KOH/g of polyesterol, particularly preferably lower than 1 mg KOH/g of polyesterol.

In the invention, the propoxylation of the polyesteretherol intermediate is carried out with use of basic catalysts. It is preferable to use aminic catalysts. Particular preference is given to catalysts selected from the group consisting of DMEOA (dimethylethanolamine), imidazole, and imidazole derivatives, in particular imidazole and imidazole derivatives, but it is also possible to use mixtures of the abovementioned catalysts to produce the propoxylated esters. Imidazole is very particularly preferably used.

The process of the invention first forms the ester structure; said ester is then propoxylated, and the molar ratio of PO to OH function of the ester here is smaller than 100, preferably smaller than 20, smaller than 5, smaller than 2, and specifically smaller than 1.5.

The ester starter is produced via condensation of the polybasic carboxylic acids and polyhydric alcohols.

The polybasic carboxylic acids used for the ester starter are selected from the group of the aromatic dicarboxylic acids, anhydrides of these, and mixtures thereof. Preference is given to aromatic dicarboxylic acids selected from the group consisting of PA (phthalic anhydride), DMT (dimethyl terephthalate), PET (polyethylene terephthalate), and TPA (terephthalic acid), and also mixtures thereof, specifically TPA-based acids (DMT, PET, TPA), and most preferably TPA.

The polyhydric alcohols used for the ester starter are mostly dialcohols having from 2 to 12 carbon atoms, for example ethylene glycol, diethylene glycol (DEG), propylene glycol, dipropylene glycol, 1,4-butanediol and its isomers, and 1,5-pentanediol and its isomers, preferably diethylene glycol.

Other starting materials can also be used to produce the ester starters, inter alia fatty acids and compounds comprising fatty acid. Examples of compounds that can be used are monocarboxylic acids, e.g. oleic acid, and oils, e.g. soy oil or castor oil.

It is also possible to use alcohols having more than two OH groups, e.g. glycerol, trimethylolpropane, or pentaerythritol, and alkoxylates of these, preferably ethoxylates. However, particular preference is given to unmodified higher-functionality components, e.g. glycerol, trimethylolpropane, or pentaerythritol, and specifically glycerol. The content of the starting materials having functionality other than 2 can be up to 60% by weight, based on the total mass of the ester starter.

The average OH functionality of the ester starter is from 1.5 to 5, preferably from 1.75 to 3, and its OH number is in the range from 50 to 400 mg/KOH/g of ester starter, preferably from 100 to 300 mg KOH/g of ester starter, and its acid number is below 5 mg KOH/g, preferably below 2 mg KOH/g, particularly preferably below 1 mg KOH/g.

In one embodiment of the process of the invention, the reaction of the polyesterol A with propylene oxide or with a mixture made of propylene oxide and ethylene oxide is carried out at a temperature in the range from 90° to 160° C., preferably from 90° to 130° C.

The invention uses propylene oxide or a mixture made of propylene oxide with ethylene oxide to alkoxylate the polyesterol A.

In one embodiment of the process of the invention, propylene oxide is used in a mixture with ethylene oxide; in one preferred embodiment, only propylene oxide is used.

The process of the invention is generally carried out at a pressure of from 0.1 to 8 bar. The addition of the alkylene oxides is usually followed by a continued-reaction phase to maximize conversion of the alkylene oxides. The resultant crude polyesterether alcohol is freed from unreacted alkylene oxide and volatile compounds by distillation, preferably in vacuo.

The formation of the adduct of the alkylene oxides with the polyesterol A to produce the desired polyesteretherol can proceed by a semibatch process or else completely continuously or semi-continuously. It is preferable to operate by the semibatch process.

In another embodiment, in addition to the starter mixture, a certain proportion of product or product precursor is charged concomitantly to the reactor (heel procedure).

Once the formation of the adduct from the alkylene oxides has been completed, the polyols are generally worked up by conventional processes, by removing the unreacted alkylene oxides, and also volatile constituents, usually by distillation, steam stripping, or gas stripping, and/or other deodorization methods. Filtration can also be carried out if required.

The OH number of the products of the process of the invention, i.e. of the polyetherester polyols which can be produced in the invention, is generally from 100 to 350 mg KOH/g, preferably from 170 to 250 mg KOH/g.

The polyetherester polyols which can be produced in the invention can be used inter alia for producing polyurethanes, in particular for producing rigid polyurethane foams, e.g. continuously- or batchwise-produced insulating sheets, sandwich elements with metallic outer layers, free-foamed slab foams, pipe insulation, etc.

The polyurethanes here, where this term is intended hereinafter to comprise polyurethanes and polyisocyanurate-modified polyurethane are produced via reaction of polyisocyanates with the polyetherester polyols which can be produced in the invention. The reaction usually takes place in the presence of blowing agents, catalysts, and conventional auxiliaries and/or additives. The information below relates to the starting materials used.

The polyisocyanates used are any of the isocyanates having two or more isocyanate groups in the molecule. It is possible here to use aliphatic isocyanates, such as hexamethylene diisocyanate (HDI) or isophorone diisocyanate (IPDI), or preferably aromatic isocyanates, such as tolylene diisocyanate (TDI), diphenylmethane diisocyanate (MDI), or a mixture made of diphenylmethane diisocyanate and polymethylene polyphenylene polyisocyanates (crude MDI). It is also possible to use the isocyanates known as modified isocyanates, where these have been modified via introduction of urethane, uretdione, isocyanurate, allophanate, uretonimine, and other groups.

Polyisocyanates a) used preferably comprise TDI or MDI, or its higher homologs, and/or its reaction products with compounds having at least two hydrogen atoms reactive toward isocyanate groups.

Production of flexible slab foams in particular uses TDI, whereas it is preferable to use MDI and its higher homologs for the preferred production of molded flexible foams and also of rigid foams.

Blowing agents used are mostly water, carboxylic acids, such as formic acid, where these form gaseous compounds during the reaction with isocyanates (these being known as chemical blowing agents), or else compounds which are gaseous at the temperature of the urethane reaction and which are inert with respect to the starting materials for the polyurethanes, and/or are known as physical blowing agents, or else a mixture thereof. Physical blowing agents mostly used are hydrocarbons having from 2 to 6 carbon atoms, halogenated hydrocarbons having from 2 to 6 carbon atoms, ketones, acetals, ethers, or inert gases, such as carbon dioxide or noble gases.

Catalysts used in the process of the invention for producing polyurethanes are preferably amine compounds and/or metal compounds, in particular heavy metal salts and/or organometallic compounds. Particular catalysts used are known tertiary amines and/or organometallic compounds, and/or carboxylates, mostly of alkali metals or ammonium ions. Examples of organometallic compounds which can be used are tin compounds, such as stannous salts of organic carboxylic acids, e.g. stannous acetate, stannous octoate, stannous ethylhexanoate, and stannous laurate, and the dialkyltin(IV) salts of organic carboxylic acids, e.g. dibutyltin diacetate, dibutyltin dilaurate, dibutyltin maleate, and dioctyltin diacetate. Examples that may be mentioend of organic amines conventionally used for this purpose are: triethylenediamine, bis(N,N-dimethylaminoethyl)ether, N,N-dimethylethanolamines, dimethyl-2-hydroxy(propyl)1,3-propylenediamine, N,N-dimethylhexadecylamine, pentamethyldipropylenetriamine, triethylamine, 1,4-diazabicyclo[2.2.2]octane, tributylamine, dimethylbenzylamine, N,N,N′,N′-tetramethylethylenediamine, N,N,N′,N′-tetramethylbutanediamine, N,N,N′,N′-tetramethylhexane-1,6-diamine, dimethylcyclohexylamine. The catalysts described are used individually or in the form of mixtures.

It is also possible to add other auxiliaries and/or additives in the process of the invention for producing polyurethanes, examples being release agents, flame retardants, dyes, fillers, and/or reinforcing agents. It is also possible to react isocyanates with the polyetheresters of the invention in blends with other polyethers or polyesters.

The polyol component, composed of the propoxylated polyester, possibly polyether, possibly unpropoxylated ester, stabilizer, possibly one or more flame retardants, and also possibly further additives, includes at least 5% by weight of the propoxylated ester which can be produced in the invention (preferably more than 10% by weight), and if the system uses no unpropoxylated ester this amount is preferably more than 10%, particularly preferably more than 20%, and specifically more than 30%, based on the entire polyol component.

In the case of production of a polyurethane (PU), the system is generally foamed with an isocyanate index >100, specifically >110, and preferably >120, and in particular instances preferably >160. In the case of production of a polyurethane comprising polyisocyanurate (PIR), the system is generally foamed with an isocyanate index >180, specifically >250, and preferably >300.

The systems can be continuous, batchwise, or involve a spray process. Preference is given to batchwise and continuous, or particularly continuous, use of the systems.

### EXAMPLES

The examples below serve to illustrate the present invention; they are not in any way to be interpreted as restricting the scope of the invention.

### Synthesis Example A

470.8 kg of a polyesterol having a hydroxyl number of 244.4 mg KOH/g determined to DIN 53240, an acid number of 0.28 mg KOH/g determined to DIN EN 14104, and a functionality of 2.3 were charged to a 600 L steel autoclave together with 165 g of imidazole, and heated to 120° C., and dried at 15 mbar for 30 minutes. 129 kg of propylene oxide were then metered into the reaction mixture within a period of 120 minutes. Once addition had ended, reaction was continued for five hours. Finally, the product was freed from volatile constituents by vacuum distillation. This gave 590 kg of the polyetherester of the invention.

Hydroxyl number (to DIN 53240): 204 mg KOH/g

Acid number (to DIN EN 14104): 0.17 mg KOH/g

Water value (to DIN 51777): 0.006%

Viscosity, 25° C. (to DIN 53 018): 6479 mPas

### Synthesis Example B

452 kg of a polyesterol having a hydroxyl number of 244.4 mg KOH/g determined to DIN 53240, an acid number of 0.28 mg KOH/g determined to DIN EN 14104, and a functionality of 2.3 were charged to a 600 L steel autoclave together with 600 g of imidazole, and heated to 120° C., and dried at 15 mbar for 30 minutes. 148 kg of propylene oxide were then metered into the reaction mixture within a period of 90 minutes. Once addition had ended, reaction was continued for eight hours. Finally, the product was freed from volatile constituents by vacuum distillation. This gave 590 kg of the polyetherester of the invention.

Hydroxyl number (to DIN 53240): 195 mg KOH/g

Acid number (to DIN EN 14104): 0.15 mg KOH/g

Water value (to DIN 51777): 0.006%

Viscosity, 25° C. (to DIN 53 018):

### Synthesis Example C

1511 kg of a polyesterol based on phthalic anhydride and diethylene glycol having an OH number of 315 mg KOH/g and a viscosity of 3000 mPa*s at 25° C. (Lupraphen® H978) were charged to a 5 L steel autoclave together with 2.5 g of imidazole, and heated to 110° C., and dried at 15 mbar for 30 minutes. 989 g of propylene oxide were then metered into the reaction mixture within a period of 180 minutes. Once addition had ended, the reaction was continued for fourteen hours. Finally, the product was freed from volatile constituents by vacuum distillation. This gave 2400 g of the polyetherester of the invention.

Hydroxyl number (to DIN 53240): 217.5 mg KOH/g

Viscosity, 25° C. (to DIN 53 018): 1202 mPas

### Synthesis Example D

1748 g of a polyesterol based on phthalic anhydride, oleic acid, trimethylolpropane, and diethylene glycol having an OH number of 240 mg KOH/g and a viscosity of 8500 mPa*s at 25° C. (Lupraphen® VP9330) were charged to a 5 L steel autoclave together with 2.5 g of imidazole, and heated to 110° C., and dried at 15 mbar for 30 minutes. 789 g of propylene oxide were then metered into the reaction mixture within a period of 60 minutes. Once addition had ended, reaction was continued for twenty hours. Finally, the product was freed from volatile constituents by vacuum distillation. This gave 2300 g of the polyetherester of the invention.

Hydroxyl number (to DIN 53240): 168 mg KOH/g

Viscosity, 25° C. (to DIN 53 018): 2171 mPas

### Counterexample A

156.9 g of a polyesterol having an OH number of 230 mg KOH/g and having an acid number of 7.3 mg KOH/g were charged to a 300 mL steel autoclave together with 0.07 g of imidazole, and heated to 130° C., and dried at 15 mbar for 60 minutes. 83.1 g of propylene oxide were then metered into the reaction mixture. After the start of the propylene oxide addition, the pressure rose continuously until a pressure maximum of 7 bar absolute was reached after two hours. No exothermic reaction was observed during the entire course of the experiment. During the phase of completion of the reaction, the pressure fell only to 5.5 bar absolute, within a period of two hours. Determination of the OH number of the reaction product confirmed the observation that reaction of the propylene oxide was incomplete. The hydroxyl number determined was 194 mg KOH/g, which deviated by 44 units from the hydroxyl number theoretically expected.

This example illustrates that the alkoxylation of a polyester having an acid number of >5 mg KOH/g does not lead to the desired product, and that the reaction does not proceed to completion, because of the deactivation of the alkoxylation catalyst.

### Comparative Example A

1846 g of a polyesterol based on phthalic anhydride and diethylene glycol having an OH number of 315 mg KOH/g and having a viscosity of 3000 mPa*s at 25° C. (Lupraphen® H978) were charged to a 5 L steel autoclave together with 2.8 g of imidazole, and heated to 130° C., and dried at 15 mbar for 30 minutes. 904 g of ethylene oxide were then metered into the reaction mixture within a period of 180 minutes. Once addition had ended, the reaction was continued for four hours. Finally, the product was freed from volatile constituents by vacuum distillation. This gave 2700 g of the polyetherester.

Hydroxyl number (to DIN 53240): 235.6 mg KOH/g

Viscosity, 25° C. (to DIN 53 018): 969.1 mPas

### Comparative Example B

2402 g of a polyesterol based on phthalic anhydride, oleic acid, trimethylolpropane, and diethylene glycol having an OH number of 240 mg KOH/g and having a viscosity of 8500 mPa*s at 25° C. (Lupraphen® VP9330) were charged to a 5 L steel autoclave together with 3.7 g of imidazole, and heated to 130° C., and dried at 15 mbar for 30 minutes. 789 g of ethylene oxide were then metered into the reaction mixture within a period of 300 minutes. Once addition had ended, reaction was continued for ten hours. Finally, the product was freed from volatile constituents by vacuum distillation. This gave 3000 g of the polyetherester.

Hydroxyl number (to DIN 53240): 186 mg KOH/g

Viscosity, 25° C. (to DIN 53 018): 995 mPas

### Comparative Example C

4431 g of terephthalic acid, 2002 g of oleic acid, 3397 g of diethylene glycol, and 1228 g are reacted in accordance with a general operating specification for producing a polyesterol. This gives a polyesterol having an OH functionality of 2.3 and having a hydroxyl number of 244.4 mg KOH/g (determined to DIN 53240) and having an acid number of 0.5 mg KOH/g (determined to DIN EN ISO 2114), which was used as comparative example in the performance tests set out below.

### Examples of the Use of the Polyetherester Polyols of the Invention:

Curing

Curing was determined by the bolt test. For this, at various junctures after mixing of the components in a polystyrene beaker, a tensile/pressure-testing machine was used to force a steel bolt with spherical cap of radius 10 mm to a depth of 10 mm into the resultant mushroom-shaped foam. The maximum force in N required here is a measure of the curing of the foam.

Flame Resistance

Flame height was measured to EN ISO 11925-2.

Adhesion:

Test specimens were produced by the twin-belt process. For this, a distance of 170 mm was set between the lower and upper belt in the twin-belt system, and test specimens were produced with a metallic outer layer. Tensile strength was then determined to DIN 53292/DIN EN ISO 527-1. If the metal sheet is peeled away from the test specimen here, the value measured corresponds to the adhesion, but if failure occurs within the foam the adhesion is greater than the value measured, which corresponds to the tensile strength of the foam.

Determination of Element Thickness

To determine element thickness after the foaming process, the distance in the twin-belt system is set to 170 mm, and sandwich elements are produced by the twin-belt process with an aluminum foil thickness of 50 pm as outer-layer material, and element thickness is determined in the middle of the element 5 minutes after production.

Production of Rigid Polyurethane Foams (Variant 1):

Production of Rigid Polyurethane Foams

The isocyanate, and also the components reactive toward isocyanate, were foamed with the blowing agents, catalysts, and all of the other additives, at a constant polyol:isocyanate mixing ratio of 100:190.

Polyol Component:

47.5 parts by weight of polyesterol as in examples or comparative examples

15 parts by weight of polyetherol having an OH number of ˜490 mg KOH/g, produced by poly-addition of propylene oxide on a sucrose/glycerol mixture as starter molecule

10 parts by weight of polyetherol composed of the ether of ethylene glycol and ethylene oxide having a hydroxy functionality of 2 and a hydroxyl number of 200 mg KOH/g

25 parts by weight of trischloroisopropyl phosphate (TCPP) flame retardant

2.5 parts by weight of Niax Silicone L 6635 stabilizer (silicone-containing stabilizer)

6.5 parts by weight of 80:20 pentane S

About 2.3 parts by weight of water

1.5 parts by weight of potassium acetate solution (47% by weight in ethylene glycol)

About 1.1 parts by weight of dimethylcyclohexylamine

Isocyanate Component:

190 parts by weight of Lupranat® M50

The mixture was then foamed in the laboratory. Envelope density was adjusted here to 38+/−1 g/L by varying the water content, at constant pentane content of 6.5 parts by weight. Fiber time was moreover adjusted to 50+/−1 s by varying the content of dimethylcyclohexylamine. Table 1 collates the results, and for purposes of illustration here the value stated for curing is the value 5 minutes after mixing of the components:

TABLE 1
Curing results

| | | Example A |
|---|---|---|
| Polyester polyol: | Comparative example C | of the invention |
| Bolt test after 5 min [N] | 79 | 86 |

Table 1 shows that the rigid polyurethane foams produced by the process of the invention harden more rapidly.

Sandwich elements of thickness 50 mm were then produced by the twin-belt process, using a twin-belt temperature of 48+/−3° C. Envelope density was adjusted here to 37+/−1 g/L by varying the water content, at constant pentane content of 6.5 parts by weight. Fiber time was moreover adjusted to 25+/−1 s by varying the content of dimethylcyclohexylamine.

Table 2 collates the results.

TABLE 2
Results of experiments in producing sandwich elements of thickness
50 mm by the twin-belt process

| Polyester polyol: | Comparative example C | Example A of the invention |
|---|---|---|
| Basal defects [%]/ | 2.8/slight defects | 0.8/perfect |
| visual assessment | | |

Table 2 shows that the quality of the rigid polyurethane foams produced by the process of the invention is higher.

Sandwich elements of thickness 170 mm were also produced by the twin-belt process with the systems comprising comparative example 2 and comprising example 2 of the invention, at a twin-belt temperature of 48+/−3° C. Envelope density was adjusted here to 37+/−1 g/L by varying the water content, at constant pentane content of 6.5 parts by weight. Fiber time was moreover adjusted to 40+/−1 s by varying the content of dimethylcyclohexylamine.

Table 3 collates the results:

TABLE 3
Results of the experiments in producing sandwich elements of
thickness 170 mm by the twin-belt process

| | | Example A |
|---|---|---|
| Polyester polyol: | Comparative example C | of the invention |
| Tensile strength to DIN | 0.05 | 0.11 |
| 53292/DIN EN ISO 527- | | |
| 1 [N] | | |
| Element thickness after | 187 mm | 179 mm |
| foaming | | |

Table 3 shows that use of the polyesterol of the invention markedly improves the dimensional stability of the polyurethane system.

### EXAMPLE

Production of Rigid Polyurethane Foams (Variant 2):

Test sheets were also produced by the twin-belt process using the following production method for a rigid polyurethane foam (variant 2).

The isocyanate, and also the components reactive toward isocyanate, were foamed with the blowing agents, catalysts, and all of the other additives, at a constant polyol:isocyanate mixing ratio of 100:170.

Polyol Component:

58 parts by weight of polyesterol as in examples or comparative examples

10 parts by weight of polyetherol composed of the ether of ethylene glycol and ethylene oxide having a hydroxy functionality of 2 and having a hydroxyl number of 200 mg KOH/g

30 parts by weight of trischloroisopropyl phosphate (TCPP) flame retardant

2 parts by weight of stabilizer; Tegostab B 8443 (silicone-containing stabilizer)

10 parts by weight of n-pentane

1.6 parts by weight of formic acid solution (85%)

2.0 parts by weight of potassium formate solution (36% by weight in ethylene glycol)

0.6 part by weight of bis(2-dimethylaminoethyl)ether solution (70% by weight in dipropylene glycol)

Isocyanate Component:

170 parts by weight of Lupranat® M50

The systems were used to produce sandwich elements of thickness 170 mm by the twin-belt process, using a twin-belt temperature of 57+/−3° C. Envelope density was adjusted here to 38+/−1 g/L by varying the pentane content, at constant formic acid content. Fiber time was moreover adjusted to 40+/−1 s by varying the content of bis(2-dimethylaminoethyl)ether (70% by weight in dipropylene glycol).

Table 4 collates the results:

TABLE 4
Results of the experiments in producing sandwich elements of
thickness 170 mm by the twin-belt process

| | | Example A |
|---|---|---|
| Polyester polyol: | Comparative example C | of the invention |
| Element thickness after | 187 mm | 179 mm |
| foaming | | |

Table 4 shows that use of the polyesterol of the invention markedly improves the dimensional stability of the polyurethane system.

Production of Rigid Polyurethane Foams (Variant 3):

Polyol Component:

36 parts by weight of polyesterol as in examples or comparative examples

30 parts by weight of polyetherol based on sucrose/glycerol having an OH number of 490 mg KOH/g

15 parts by weight of glycol mixture made of DPG and MPG (1:1)

14 parts by weight of polypropylene glycol having an OH number of 104 mg KOH/g

3 parts by weight of glycerol

2 parts by weight of Tegostab B 8462 silicone-containing stabilizer

1.5 parts by weight of water

0.01 part by weight of dimethylcyclohexylamine

Isocyanate Component:

165 parts by weight of polymeric methylenediphenyl diisocyanate, NCO content ˜31.5% by weight, viscosity ˜200 mPa*s (25° C.)

Production:

The isocyanate, and also the components reactive toward isocyanate, were mixed together with the blowing agents, catalysts, and all of the further additives, using an NCO index of 125, and the reaction mixture was poured into a box of basal area 225 mm×225 mm, where it was foamed. Foam density of 100 g/L and fiber time of 360 sec were kept constant via appropriate adjustment of the amount of water and the amount of amine. To produce the reinforced rigid foams, reaction mixture was charged to the same box, but this now comprised seven layers of Unifilo U809-450 glass fiber mats from OCV.

Determination of Penetration Time:

Markings were made at 5 points on the uppermost glass fiber mat. The penetration time began as soon as the reaction mixture was poured onto the 7 layers of glass fiber mats, and it ended as soon as at least 4 of the 5 marked points had become visible again.

Determination of Compatibility Between Glass Fibers and PU:

Once the foam cube had hardened, it was divided perpendicularly to the glass fiber mats, and the distances of the adjacent glass fiber mats from one another were determined. This information was used to calculate the average distance between the mats, and also the attendant standard deviation.

TABLE 5

| | Comparative | Comparative | Example 4 |
|---|---|---|---|
| | example 4 | example 5 | of the invention |
| Polyesterol | Ester starter* | Ester | Ester |
| | | starter:Comparative | starter:Synthesis |
| | | example A = 1:1 | example C = 1:1 |
| Viscosity of | 1400 | 1230 | 1250 |
| A component | | | |
| (mPa * s, at | | | |
| 25° C.) | | | |
| Penetration | 99 | 96 | 85 |
| time, sec | | | |
| Foam quality | good | good | good |
| Standard | 0.41 | 0.31 | 0.32 |
| deviation | | | |
| of distance | | | |
| between | | | |
| mats (cm) | | | |
| Compressive | 0.93/24 | n.d. | 0.90/24 |
| strength/ | | | |
| compressive | | | |
| modulus of | | | |
| elasticity | | | |
| (N/mm²) | | | |
| Tensile strength/ | 1.0/34 | n.d. | 1.0/33 |
| tensile | | | |
| modulus of | | | |
| elasticity | | | |
| (N/mm²) | | | |

*Ester starter: Polyesterol based on phthalic anhydride and DEG, OH number 315 mg KOH/g, viscosity 3000 mPa * s (25° C.)

Table 5 shows that alkoxylation makes the A components less viscous and increases the homogeneity of mat distribution in the foam. Only propoxylation leads to the accelerated penetration times. There is no impairment of mechanical properties here. Production can therefore be accelerated markedly with propoxylated esters, with a slight improvement in product quality.

Production of a Rigid Polyurethane Foam (Variant 4):

Polyol Component:

23 parts by weight of polyesterol 1 based on terephthalic acid, oleic acid, TMP, and DEG, OH number 245 mg KOH/g

23.5 parts by weight of polyesterol 2 as in examples or comparative examples

25 parts by weight of polyetherol based on sorbitol having an OH number of 490 mg KOH/g

6 parts by weight of polyethylene glycol having an OH number of 200 mg KOH/g

20 parts by weight of TCPP

2.5 parts by weight of Niax Silicone L-6635 silicone-containing stabilizer

1.4 parts by weight of water

7 parts by weight of n-pentane

1.5 parts by weight of potassium acetate solution (47% by weight in ethylene glycol)

0.7 part by weight of amine catalyst (mixture of tertiary amines)

Isocyanate Component:

About 165 parts by weight of polymeric methylenediphenyl diisocyanate, NCO content ˜31.5% by weight, viscosity ˜500 mPa*s (25° C.)

Production: The isocyanate, and also the components reactive toward isocyanate, were mixed together with the blowing agents, catalysts, and all of the further additives, using an NCO index of 190, and the reaction mixture was poured into a box of basal area 225 mm×225 mm, where it was foamed. Foam density of 45 g/L and fiber time of 45 sec were kept constant via appropriate adjustment of the amount of water and the amount of amine.

TABLE 6

| | | | Example 5 |
|---|---|---|---|
| | Comparative | Comparative | of the |
| | example 6 | example 7 | invention |
| Polyesterol 2 | Ester starter* | Counter | Synthesis |
| | | example B | example D |
| Viscosity of A component | 2500 | 1500 | 1800 |
| (mPa * s, at 25° C.) | | | |
| Bolt test after 5 min | 111 | 116 | 116 |
| (N) | | | |
| 3-point flexural | 0.36 | not known | 0.37 |
| strength (N/mm²) | | | |
| Deflection at break | 5.9 | not known | 7.1 |
| (mm) | | | |

*Ester starter: Polyesterol based on phthalic anhydride, oleic acid, TMP, and DEG; OH number 240 mg KOH/g, viscosity 8500 mPa * s (25° C.)

No impairment of the hardening process, but the foam is less brittle and breaks only when subjected to markedly greater deflection.

Production of Rigid Polyurethane Foam (Variant 5):

Polyol Component:

22.5 parts by weight of polyesterol 1 based on terephthalic acid, oleic acid, TMP, and DEG, OH number 245 mg KOH/g

22.5 parts by weight of polyesterol 2 as in examples or comparative examples

12 parts by weight of polyethylene glycol having an OH number of 190 mg KOH/g

40 parts by weight of a mixture of aliphatic phosphoric esters

3 parts by weight of Tegostab B 8467 silicone-containing stabilizer

3.5 parts by weight of formic acid solution (85% by weight in water)

13 parts by weight of n-pentane

2 parts by weight of potassium formate solution (36% by weight in ethylene glycol)

2.2 parts by weight of amine catalyst (mixture of tertiary amines)

Isocyanate Component:

About 320 parts by weight of polymeric methylenediphenyl diisocyanate, NCO content ˜31.5% by weight, viscosity ˜500 mPa*s (25° C.)

Production:

The isocyanate, and also the components reactive toward isocyanate, were mixed together with the blowing agents, catalysts, and all of the further additives, using an NCO index of 500, and the reaction mixture was poured into a box of basal area 225 mm×225 mm, where it was foamed. Foam density of 40 g/L and fiber time of 50 sec were kept constant via appropriate adjustment of the amount of formic acid and the amount of amine.

TABLE 7

| | Comparative | Comparative | Example 6 of the |
|---|---|---|---|
| | example 8 | example 9 | invention |
| Polyesterol 2 | Starter | Counter | Synthesis |
| | polyester* | example B | example D |
| Viscosity of | 700 | 350 | 450 |
| A component | | | |
| (mPa * s, at 25° C.) | | | |
| Bolt test after 4 min (N) | 90 | 91 | 86 |
| BKZV (cm) | 6.7 | 6.7 | 6.8 |
| 3-point flexural strength | 0.21 | 0.19 | 0.24 |
| (N/mm²) | | | |
| 3-point flexural modulus | 2.6 | 2.6 | 3.1 |
| (N/mm²) | | | |
| Deflection at break (mm) | 5.0 | 5.5 | 6.2 |

*Starter polyester: Polyesterol based on phthalic anhydride, oleic acid, TMP, and DEG; OH number 240 mg KOH/g, viscosity 8500 mPa * s (25° C.)

Flame height remains identical at high NCO index. Hardening also remains equally good. Only the propoxylated polyester makes the foam less brittle, as can be seen from the 3-point flexural data: strength, modulus, and maximum deflection increase.

In summary, the examples therefore reveal that the propoxylation of polyesters can reduce brittleness and can improve adhesion, surprisingly without impairing other properties, in particular flame retardancy. This advantageous effect occurs only on propoxylation. Analogous ethoxylation leads to markedly smaller or sometimes adverse effects in the foam.

Examples moreover reveal that the hardening of the foam can be improved, giving elements of greater dimensional stability.

The compatibility of the polyurethane with glass fiber mats is moreover improved, as measured by accelerated penetration time for a reaction mixture through layers of glass fiber mats.

1. A process for producing a polyesteretherol, where at least one compound selected from the group consisting of aromatic dicarboxylic acids, anhydrides of these, and mixtures thereof is first reacted with at least one compound from the group of the polyhydric alcohols to form a polyesterol A with an acid number smaller than 5 mg KOH/g, and then, with use of a basic catalyst, the resultant polyesterol A is reacted with propylene oxide or with a mixture made with propylene oxide and ethylene oxide.

2. The process for producing a polyesteretherol according to claim 1, where only propylene oxide is used.

3. The process for producing a polyesteretherol according to claim 1, where a mixture made of propylene oxide and ethylene oxide is used.

4. The process for producing a polyesteretherol according to claim 1, where the reaction of the polyesterol A with propylene oxide or with a mixture made of propylene oxide and ethylene oxide is carried out at a temperature in the range from 90° C. to 160° C.

5. The process for producing a polyesteretherol according to any of claims 1 to 4, where the basic catalyst is an amine compound.

6. The process for producing a polyesteretherol according to any of claims 1 to 5, where the basic catalyst has been selected from the group consisting of DMEOA (dimethylethanolamine), imidazole and imidazole derivatives, and mixtures made of the above-mentioned.

7. The process for producing a polyesteretherol according to claim 6, where the catalyst is imidazole.

8. The process for producing a polyesteretherol according to any of claims 1 to 7, where the molar ratio of propylene oxide to the number of OH functions of the polyesterol is smaller than 100.

9. The process for producing a polyesteretherol according to any of claims 1 to 8, where, to produce the polyesterol A, at least one compound selected from the group consisting of PA (phthalic anhydride) and TPA (terephthalic acid) is used.

10. The process for producing a polyesteretherol according to any of claims 1 to 9, where, to produce the polyesterol A, glycerol and/or DEG (diethylene glycol) is used as polyhydric alcohol.

11. The process for producing a polyesteretherol according to any of claims 1 to 10, where, to produce the polyesterol A, a fatty acid or a compound comprising fatty acid is also used.

12. A polyesteretherol which can be produced by the process of any of claims 1 to 11.

13. The use, for producing a polyurethane, of a polyesteretherol which can be produced according to claims 1 to 11.

14. A rigid polyurethane foam obtainable via mixing of
(a) isocyanate with
(b) compounds having groups reactive toward isocyanates, and
(c) blowing agent, and
(d) flame retardant, and
(e) catalysts, and
(f) further additives,
where the isocyanate (a) is based on polymeric MDI and has a viscosity at 25° C. of from 100 to 3000 mPa*s, preferably from 400 to 600 mPa*s, and an NCO content of from 27 to 34% by weight, preferably from 29 to 32% by weight,
the compounds (b) having groups reactive toward isocyanates comprise a polyesteretherol (b1) which can be produced by the process of any of claims 1 to 11, a polyesterol (b2) having a functionality of less than or equal to 3, a polyetherol (b3) having a functionality greater than 2, a polyethylene glycol (b4) having molar mass smaller than 1000 g/mol, and optionally chain extenders (b5) and crosslinking agents (b6), where the proportion of the polyesteretherol (b1) and polyesterol (b2) together is from 10 to 80% by weight, of the polyetherol (b3) is from 0 to 80% by weight, of the polyethylene glycol (b4) is from 0 to 25% by weight, and of the chain extender (b5) and crosslinking agent (b6) together is from 0 to 20% by weight, based in each case on the total weight of the compounds (b) having groups reactive toward isocyanates, where the proportion of the polyols (b1), (b2), (b3), (b4), (b5), (b6) in the compounds (b) having groups reactive toward isocyanates is preferably 100% by weight, based on the total weight of the compounds (b) having groups reactive toward isocyanates, and where the proportion of the polyesteretherol (b1), based on the total weight of the compounds (b) having groups reactive toward isocyanates, is at least 5% by weight,
the blowing agent (c) is composed of at least 50% by weight of hydrocarbons, particularly preferably of at least 50% by weight of cyclopentane, n-pentane, isopentane, or a mixture of these, in particular of at least 50% by weight of n-pentane, based on the total weight of blowing agent (c), and the total amount of blowing agent (c), based on the rigid polyurethane foam is such that the average density achieved in the rigid polyurethane foam is in the range from 25 to 100 g/L, preferably from 30 to 50 g/L, and the flame retardant (d) preferably comprises at least one phosphoric ester, and the proportion of the flame retardant (d) is from 0 to 40% by weight, based on the total weight of (b)+(d).

15. A rigid polyurethane foam obtainable via mixing of
(a) isocyanate
(b) compounds having groups reactive toward isocyanates,
(c) blowing agent
(d) catalysts, and
(e) further additives,
where the isocyanate (a) is based on polymeric MDI and has a viscosity at 25° C. of from 100 to 3000 mPa*s, preferably from 100 to 600 mPa*s, and an NCO content of from 27 to 34% by weight, preferably from 29 to 32% by weight,
the compounds (b) having groups reactive toward isocyanates comprise a polyesteretherol (b1) which can be produced by the process of any of claims 1 to 11, a polyesterol (b2) having a functionality of less than or equal to 3, a polyetherol (b3) having a functionality of at least 4 and having a viscosity at 25° C. of at most 10 000 mPa*s, a polyetherol (b4) having a functionality of at most 3 and having a viscosity at 25° C. of at most 600 mPa*s, a chain extender (b5), and optionally a crosslinking agent (b6), where the proportion of the polyesteretherol (b1) and polyesterol (b2) together is from 20 to 60% by weight, of the polyetherol (b3) is from 20 to 50% by weight, of the polyetherol (b4) is from 0 to 30% by weight, of the chain extender (b5) is from 5 to 20% by weight, and of the crosslinking agent (b6) is from 0 to 10% by weight, based in each case on the total weight of the compounds (b) having groups reactive toward isocyanates, where the proportion of the polyols (b1), (b2), (b3), (b4), (b5), (b6) in the compounds (b) having groups reactive toward isocyanates is preferably 100% by weight, based on the total weight of the compounds (b) having groups reactive toward isocyanates, and where the proportion of the polyesteretherol (b1), based on the total weight of the compounds (b) having groups reactive toward isocyanates, is at least 5% by weight,
the total amount of blowing agent (c), based on the rigid polyurethane foam, is such that the density achieved in the rigid polyurethane foam is in the range from 50 to 200 g/L, preferably from 80 to 120 g/L,
and the additives (e) comprise a reinforcing agent, preferably composed of glass fiber mats, where the amount used of the reinforcing agent is from 5 to 15% by weight, based on the total weight of the rigid polyurethane foam inclusive of reinforcing agent.

16. The use, for producing a polyurethane according to claim 14 or 15, of a polyesteretherol which can be produced according to claims 1 to 11, where the production of the polyurethane uses a polyisocyanate, and where the isocyanate index is >100.
